(19) United States
(12) Patent Application Publication
(10) Pub. No.: US 2026/0043761 A1
(43) Pub. Date: Feb. 12, 2026

(54) Method and System for Determining Similarity by Comparing 1H-NMR and 1H-1H Cosy NMR Spectra

(71) Applicant: LG Chem, Ltd., Seoul (KR)

(72) Inventors: Seong In Jo, Daejeon (KR); Kyu Hwang Lee, Daejeon (KR); Jae Hyun Kim, Daejeon (KR); Sae Bo Mi Park, Daejeon (KR); Eun Hee Kim, Daejeon (KR); Jeong Min Oh, Daejeon (KR); Yong Jin Bae, Daejeon (KR)

(73) Assignee: LG Chem, Ltd., Seoul (KR)

(21) Appl. No.: 18/998,899

(22) PCT Filed: Jan. 10, 2024
(86) PCT No.: PCT/KR2024/000498

§ 371 Date: Jan. 27, 2025

(30) Foreign Application Priority Data
Jan. 11, 2023 (KR) 10-2023-0004075
Jan. 11, 2023 (KR) 10-2023-0004076
Jan. 9, 2024 (KR) 10-2024-0003693

(51) Int. Cl. G01N 24/08; G01R 33/46; G16C 20/20; G16C 20/40; G16C 20/70

(52) U.S. Cl. CPC G01N 24/087; G01R 33/4625; G16C 20/20; G16C 20/40; G16C 20/70

(57) ABSTRACT

A system and method for estimating a molecular structure by verifying structural features included in a molecular structure as structural features of a candidate molecular structure is provided The system and method compare an NMR spectrum of a target substance with spectra of candidates generated from the verified candidate molecular structure to determine a similar candidate molecular structure based on the-spectrum information.

### CROSS-REFERENCE TO RELATED APPLICATIONS

The present application is a national phase entry under 35 U.S.C. § 371 of International Application No. PCT/KR2024/000498 filed Jan. 10, 2024, published in Republic of Korea, which claims priority from Korean Patent Application No. 10-2023-0004075 filed on Jan. 11, 2023; Korean Patent Application No. 10-2023-0004076 filed on Jan. 11, 2023; and Korean Patent Application No. 10-2024-0003693 filed on Jan. 9, 2024, all of which are incorporated herein by reference.

### TECHNICAL FIELD

The present disclosure relates to a technique for enabling a researcher to efficiently estimate the molecular structure of an unknown substance by calculating a plurality of candidate molecular structures and extracting unsuitable candidate molecular structures from the plurality of candidate molecular structures to reduce the number of candidate molecular structures.

In addition, the present disclosure relates to a technique for extracting a final candidate molecular structure from the reduced number of candidate molecular structures through ¹H-NMR spectrum analysis and comparison.

### BACKGROUND

1H-NMR spectrum analysis is performed to estimate the molecular structure of an unknown substance. At this time, an analyst derives all possible candidate molecular structures from an acquired 1H-NMR spectrum, and predicts peak positions and shapes of all hydrogens in each candidate molecular structure to create a virtual spectrum. Thereafter, the most reasonable structure is selected by comparing the virtual spectrum with the actual spectrum (in terms of notation, in the present disclosure, 1H-NMR and ¹H-NMR, 1H-1H NMR and ¹H-¹H COSY NMR are regarded the same, respectively).

However, when a large number of peaks are concentrated in a small region, as in the case of an aromatic macromolecule, there is a problem in that the above-described method is difficult for use in comparing similar molecules.

For example, the following background reference does not use information such as peak multiplication in a spectrum, and thus, is not capable of reflecting detailed structural information, which results in no deriving an accurate molecular structure estimation result. A Structure Elucidation System Based on ¹H NMR and H—H COSY Spectra in Organic Chemistry, Hideyuki Masui and Huixiao Hong, J. Chem. Inf. Model. 2006, 46, 2.

### DISCLOSURE

### Technical Problem

The present disclosure aims to solve the problem of the prior art and to provide a method and a system capable of performing accurate 1H-NMR spectrum analysis even for a substance having a large molecular size or a complex shape, such as an aromatic ring.

### Technical Solution

In order to solve the above-mentioned technical problem, the present disclosure provides a method for predicting a molecular structure of a target substance from a 1H-NMR spectrum of the target substance, wherein the method includes a key spectrum acquisition procedure for acquiring a 1H-NMR spectrum of a target substance, a candidate spectrum information acquisition procedure for acquiring spectrum information of a virtual spectrum of each candidate substance from candidate molecular structures of the target substance as candidate spectrum information, a spectrum comparison procedure for comparing the key spectral information with the candidate spectrum information to calculate similarity between the key spectrum and the candidate spectrum; and a target substance molecular structure determination procedure for deriving a candidate spectrum with the highest similarity in the spectrum comparison procedure, and selecting the molecular structure of a corresponding candidate substance as the molecular structure of the target substance.

In addition, a method for determining similarity by comparing two NMR spectra, wherein the method includes a peak value information extraction step of extracting peak value information for spectrum comparison from each peak of a first spectrum as a key spectrum and a second spectrum as a candidate spectrum, a peak correspondence step of matching each peak of the first spectrum and the second spectrum; a spectrum similarity calculation step of calculating similarity between the first spectrum and the second spectrum by comparing the peak value information of the peaks of the first spectrum and the second spectrum matched.

Furthermore, the present disclosure provides a system for estimating a molecular structure of a target substance, wherein the system includes a spectrum acquisition device that acquires a 1H-NMR spectrum and a 1H-1H NMR spectrum of a target substance from an NMR device as a key spectrum of the target substance, a candidate molecular structure acquisition module that acquires candidate molecular structures of the target substance and calculates a candidate molecular structure list, a virtual spectrum calculation module that calculates a virtual spectrum as a candidate spectrum from the candidate molecular structures included in the candidate molecular structure list, a spectrum information extraction module that extracts spectrum information from each of the key spectrum and the candidate spectrum, a spectrum similarity calculation module that calculates similarity between the key spectrum and the candidate spectrum by using the spectrum information of the key spectrum and the candidate spectrum, a final estimated structure output module that outputs a candidate molecular structure corresponding to a candidate spectrum with the highest similarity as a final estimated molecular structure of the target substance.

In addition, the present disclosure extracts hydrogen structural features that identify hydrogens having a predetermined structural feature from the candidate molecular structures, and through verification of suitability of candidate molecular structures, which includes a procedure of verifying whether all hydrogens having the predetermined structural feature may be assigned to all peaks of the 1H-NMR spectrum, verifies each candidate molecular structure to update a candidate molecular structure list excluding unsuitable candidate molecular structures, thereby reducing the number of candidate molecular structures to increase computational efficiency.

To this end, a system for estimating a molecular structure of a target substance according to the present disclosure includes a hydrogen structural feature identification module of identifying hydrogen atoms having a predetermined structural feature in each candidate molecular structure of the target substance, a peak assignment validity determination module that verifies, based on the structural feature information of each hydrogen identified from the candidate molecular structure, whether each hydrogen of the candidate molecular structure may be assigned to all peaks in the spectrum, a valid candidate molecular structure output module that updates the candidate molecular structure list by using results of the peak assignment validity judgment module.

### Advantageous Effects

The present disclosure provides a method for calculating similarity of two spectra by using 2D NMR spectrum information as well as 1D NMR spectrum information to bring an effect of accurately and efficiently estimating a molecular structure of a target substance by comparing a spectrum of the target substance and a spectrum of a candidate substance.

In addition, the present disclosure quickly excludes unsuitable candidate substances from candidate molecular structures, and thus, brings an effect of estimating an accurate molecular structure of an unknown substance more quickly through similarity calculation.

### BRIEF DESCRIPTION OF THE DRAWINGS

The following drawings attached herein illustrate preferred aspects of the present disclosure by example, and with the above-described detailed description of the present disclosure, serve to enable technical concepts of the present disclosure to be further understood, and therefore, the present disclosure should not be construed as limited to only the matters described in such drawings.

FIG. 1 is an example of a COSY spectrum used in the present disclosure.

FIG. 2 is a flowchart showing a procedure for calculating spectrum similarity and estimating a molecular structure according to the present disclosure.

FIG. 3 is a block diagram of a system for calculating spectrum similarity and estimating a molecular structure of the present disclosure.

### DETAILED DESCRIPTION

Reference numerals used in the present disclosure are as follows:
10 Spectrum acquisition device;
20 Spectrum information extraction module;
30 Candidate molecular structure acquisition module;
40 Virtual spectrum calculation module;
50 Spectral similarity calculation module;
60 Final estimated structure output module;
70 Deuterium substitution position calculation module
80 Hydrogen structural feature extraction module;
90 Peak assignment validity determination module; and
100 Valid candidate molecular structure output module.

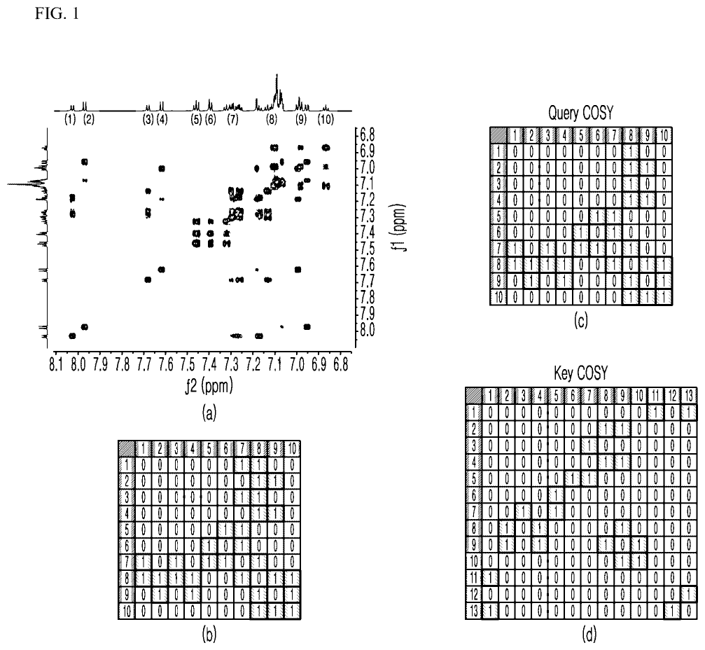

The present disclosure uses a 1H-1H COSY spectrum as one of a 1H-NMR spectrum and a 2D NMR spectrum of a substance. The 1H-1H COSY spectrum is obtained from a nuclear magnetic resonance spectrometer (NMR). Both a horizontal axis and a vertical axis are composed of a 1H spectrum in the 1H-1H COSY spectrum, and FIG. 1 shows an example thereof. In the present disclosure, a 1HNMR spectrum, a ¹H-NMR spectrum, a 1H-1H COSY NMR spectrum, and a 1H-1H COSY NMR spectrum each denote the same thing.

### 1. Method for Estimating Molecular Structure of Target Substance According to the Present Disclosure

A method for estimating the molecular structure of a target substance according to the present disclosure acquires spectrum (this is called a virtual spectrum) information corresponding to a plurality of candidate substance molecular structures, and then compares virtual spectrum information of a candidate substance with spectrum (this is called a key spectrum) information of a target substance to select a virtual spectrum that is most similar to the key spectrum, and selects the molecular structure of the corresponding candidate substance as the molecular structure of the target substance.

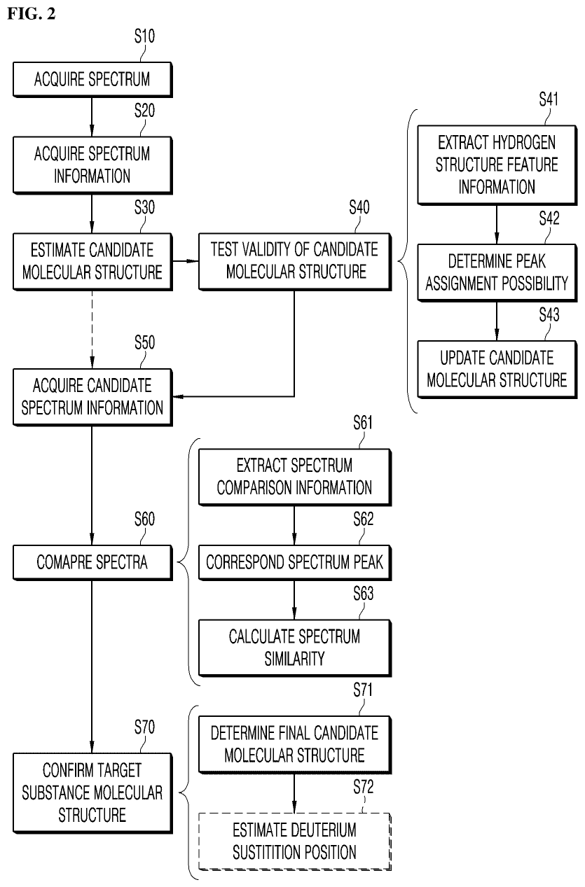

The procedure according to the present disclosure is largely composed of the following three procedures. First, A. a procedure of acquiring the spectrum information of a target substance for which the molecular structure is to be estimated, followed by B. a procedure of selecting candidate molecular structures of the target substance, and then C. a procedure of comparing virtual spectra of the candidate molecular structures with a spectrum of the target substance to acquire a final molecular structure of the target substance from the candidate molecular structures. Hereinafter, each procedure will be described in more detail with reference to FIG. 2.

### A. Procedure of Acquiring Spectrum Information of Target Substance

### (1) Key Spectrum Acquisition Step (S10)

This is a procedure of acquiring a 1H-NMR spectrum and a 1H-1H COSY spectrum of a target substance from an NMR. The 1H-NMR spectrum and the 1H-1H COSY spectrum acquired are called a key spectrum.

### (2) Key Spectrum Information Acquisition Step (S20)

Spectrum information is acquired from the key spectrum. The spectrum information includes the following information.
(a) Peak position (chemical shift value): x-axis value of each peak in one-dimensional spectrum
(b) Peak split value (multiplicity (split)): Number of small peaks forming each peak (for example, in the spectrum of FIG. 1, the split of the leftmost peak is 2, and the split of the rightmost peak is 3)
(c) Peak integral value: Area value of each peak
(d) COSY information (COSY): Information of all peak pairs in which COSY peaks are observed.

Example) In the two-dimensional key spectrum of FIG. 1, a red dot is displayed at a peak position related to the first peak on the left, and this is called a COSY peak. Since the first peak showed a COSY peak with the seventh and eighth peaks, for a matrix with rows and columns that are respectively a peak list, an element at a (1, 7), (1, 8) position is set to 1.

FIG. 1 is a diagram showing a predetermined two-dimensional key spectrum (a) and a COSY matrix (b) displaying information of a COSY peak thereof, and in (a) of FIG. 1, if there is a COSY peak present between two 1D peaks, an element value of the COSY matrix of (b) in FIG. 1 is set to 1. In the example above, since the first peak (1) shows the COSY peaks with the seventh and eighth peaks (7) and (8), it can be seen that the element values of COSY (1, 7) and COSY (1, 8) are each set to 1 in the COSY matrix of FIG. 1(b).

### B. Procedure of Selecting Candidate Molecular Structures of Target Substance

A procedure of selecting candidate molecular structures of the target substance will be described.

### (1) Procedure of Acquiring Candidate Molecular Structures (S30)

This is a step of acquiring candidate molecular structure information of the target substance. A candidate molecular structure is estimating a molecular structure of a candidate that the target substance may have, in order to finally estimate what kind of molecular structure the target substance has. By using measurement information of the target substance, such as mass spectrometry for the target substance, it is possible to estimate information of a plurality of candidate molecular structures, which could be the molecular structure of the target substance.

The plurality of candidate molecular structures are acquired by an analysis method such as LC-MS/MS, and for example, a candidate molecular structure may be acquired by acquiring a molecular weight and a molecular formula of the target substance, and then searching a molecular structure having the same molecular formula in a known substance database (SCIFinder, PubChem, etc.). Another example is a method for creating possible candidate molecular structures by acquiring partial structure information of a molecule from MS/MS analysis results and then assembling the partial structure information.

A list of candidate molecular structures including the plurality of candidate molecular structures of the target substance acquired as described above is created.

### (2) Procedure of Testing Validity of Candidate Molecular Structures (S40)

A molecular structure of the target substance may be acquired through spectrum comparison of each spectrum of the above-acquired candidate molecular structures of the target substance according to a procedure of acquiring a final molecular structure of the target substance from the candidate molecular structures, which is described below in Procedure C.

However, in this case, spectra of numerous candidate molecular structures should be compared, so that to reduce the number of candidate molecular structures, a procedure for verifying suitability of candidate molecular structures may be performed prior to the spectrum comparison procedure.

The candidate molecular structure validity procedure extracts feature information of hydrogen from a plurality of candidate molecular structures, determines validity of non-suitable candidate molecular structures based on whether all hydrogens of each candidate molecular structure may be assigned to all peaks in a spectrum of the target substance, and excludes the non-suitable candidate molecular structures from the list of candidate molecular structures. This procedure follows detailed procedures below.

### (2-1) Hydrogen Structural Feature Information Extraction Step (S41)

This is a step of identifying structural feature information of hydrogens constituting each candidate molecular structure included in the list of candidate molecular structures to extract hydrogen feature information of each candidate molecular structure. Hydrogen feature information to be identified includes the following:

### \textcircled{1} Number of Isomorphic Hydrogen

It refers to the number of hydrogens having a bonding structure that is structurally isomorphic with that of a target hydrogen. The number of isomorphic hydrogens becomes an integral value of a corresponding hydrogen peak.

\textcircled{2} Number of Hydrogens Linked to 1st-Neighbor Atom

It is the number of hydrogens linked to a 1st-neighbor atom among atoms linked to the target hydrogen. Multiplicity of the target hydrogen is determined from the number of 1st-neighbor atom-linked hydrogens linked to the 1st-neighbor atom.

\textcircled{3} Number of Hydrogens Linked to 2nd-Neighbor Atom

It is the number of hydrogens linked to a second linked atom via the 1st-neighbor atom among atoms linked to the target hydrogen. That is, it is the number of hydrogens linked to an atom directly linked to the 1st-neighbor atom. This affects the multiplicity of the target hydrogen, and is used to predict the shape of a peak.

### \textcircled{4} Other Hydrogens That May Have COSY Peak

Other hydrogens that may have the same COSY peak as that of the target hydrogen are identified. If the distance between two hydrogens is within a specific bonding distance, a COSY signal is displayed.

As such, by extracting the structural feature information of each hydrogen from each candidate molecular structure, it is determined whether it is possible to assign each hydrogen of each candidate molecular structure to each peak of the key spectrum of the target substance based on the feature information of the hydrogen of each candidate molecular structure in the next step.

### (2-2) Peak Assignment Possibility Determination Step (S42)

This is a step of determining whether all hydrogens of a given candidate molecular structure may be assigned to all peaks of the key spectrum.

If all hydrogens of the given candidate molecular structure cannot be assigned to all peaks in the key spectrum, a molecular structure validity test according to the present disclosure determines that the corresponding candidate molecular structure is an invalid molecular structure.

Therefore, in order to determine if assignment is impossible, all possible assignment methods should be reviewed, which requires a very large amount of calculation. For example, if a predetermined candidate molecular structure includes m distinguishable hydrogens, and there are n peaks in a spectrum, there are mPn possible assignment methods, so that the time to determine assignment suitability increases exponentially according to molecular complexity.

Accordingly, the present disclosure determines the possibility of assignment by replacing a problem of assigning all hydrogens of the candidate molecular structure to each peak of the key spectrum with a mixed integer linear programming (MIP) problem having linear constraints.

Here, since there is no optimization target for the MIP problem, slack variables, s, and t are introduced, and the possibility of assignment is determined based on the success/failure of optimization itself.

An equation for optimization is Equation 1 below, and the optimization is to obtain optimal solutions si and ti that minimize Equation 1.

$$minimize {\sum }_{i}^{N} ( {s}_{i} + {t}_{i} ) \qquad [ Equation 1 ]$$

for M×N sized matrix X
(If hydrogen j is not assigned to peak i, Xᵢⱼ=0, if hydrogen j is assigned to peak i, Xᵢⱼ=1, j=1, 2, . . . , M, i=1, 2, . . . , N)

### <Constraints>

Constraints for the calculation of Equation 1 are as follows.
\textcircled{1} sᵢ≥0, tᵢ≥0 for 1≤i≤N
\textcircled{2} For all 1≤i≤N, the peak integral value of hydrogen j is intg_{hydrogen}(j) and the integral value of peak i of the key spectrum is intgₚₑₐₖ(i),

$${s}_{i} = {intg}_{peak} ( i ) - {\sum }_{j}^{M} {X}_{ij} \cdot {intg}_{hydrogen} ( j ) .$$

$${t}_{i} = {intg}_{peak} ( i ) + {\sum }_{j}^{M} {X}_{ij} \cdot {intg}_{hydrogen} ( j )$$

\textcircled{3} Xᵢⱼ=0, (if the multiplicity of hydrogen j is different from the multiplicity of peak i)
\textcircled{4} Xᵢⱼ=0, (if the peak integral value of hydrogen j is greater than the integral value of peak i)
\textcircled{5} For two peaks i_1 and i_2 having COSY peaks, there should be at least one COSY peak present between all hydrogen sets C_hydrogen(i1) assigned to the i_1 and all hydrogen sets C_hydrogen(i2) assigned to the i_2.

### (2-3) Candidate Molecular Structure List Update Step (S43)

In the optimized Equation 1, if the optimization is successful and the optimal solutions for s and t are obtained, all hydrogen of the corresponding structure may be assigned to peaks of the key spectrum, and this is determined to be a valid structure. If the optimization fails and no optimal solution is obtained, it is considered that there are one or more unassignable hydrogens present, so that it is determined that the corresponding candidate structure is a candidate molecular structure not suitable for a given spectrum.

In this step, as such, unsuitable candidate molecular structures are excluded and the list of candidate molecular structures is updated to output a list of valid candidate molecular structures.

### C. Final Molecular Structure Selection Step

This is a procedure of comparing spectrum information of candidate molecular structures included in the obtained candidate molecular structure list or in the valid candidate molecular structure list with the spectrum information of the target substance to select the final molecular structure of the target substance. This is accomplished through the detailed procedures below.

### (1) Candidate Spectrum Information Acquisition Procedure (S50)

A virtual spectrum corresponding to each candidate molecular structure is acquired from the above-acquired information of the plurality of candidate molecular structures, which is set as a spectrum of each candidate molecular structure, and information of each spectrum is acquired. The virtual spectrum of each candidate molecular structure is acquired using a known virtual spectrum information calculation device or software for calculating virtual spectrum information when molecular structure information is input.

Candidate spectrum information, which is spectrum information of a candidate spectrum for a candidate molecular structure to be obtained, includes items such as the key spectrum information described above.

### (2) Spectrum Comparison Procedure (S60)

By comparing the spectrum information (each peak value data) of the key spectrum, which is the spectrum of the target substance, with the spectrum information of each candidate, similarity between the key spectrum and the candidate spectrum is calculated. A spectrum comparison method according to the present disclosure to be described below is applied to the spectrum comparison.

A virtual spectrum of a candidate substance, which is most similar to the key spectrum, is derived according to a spectrum similarity calculation procedure to be described below.

A procedure of determining similarity by comparing NMR spectra of two molecules according to the present disclosure will be described.

### (2-1) Spectrum Comparison Information Extraction From Key Spectrum and Candidate Spectrum (S61)

Comparison information for spectrum comparison is extracted from each peak of the key spectrum and the candidate spectrum. The comparison information is the spectrum information described above.

### (2-2) Peak Correspondence Step of Matching Peak of Candidate Spectrum to Peak of Key Spectrum (S62)

This is a step of assigning a peak Qi of a candidate spectrum to a peak Ki of the key spectrum. Here, the i is a peak number of the key spectrum and the candidate spectrum.

When matching the Qi to the Ki, the following rules should be satisfied.
\textcircled{1} An integral value of Qi should not be greater than an integral value of Ki.
\textcircled{2} A split value of Qi should be the same as a split value of Ki.
\textcircled{3} One or more hydrogens having a COSY relationship with Qi should be assigned to other peaks having a COSY relationship with Ki.

COSY peak assignment of the query peak Qi and the key peak Ki will be described with reference to (c) and (d) of FIG. 1.

If a COSY matrix of the key spectrum and a COSY matrix of the query or candidate spectrum are obtained as shown in (c) and (d) of FIG. 1, the constraints mean that when there is a COSY peak between the key peaks i1 and i2, there should be at least one reciprocal COSY peak present between a query peak set C_query(i1) assigned to i1 and a query peak set C_query(i2) assigned to i2.

Considering a case in which two of K11 and K13 are assigned to a Q7 peak, and three of K8, K9, and K10 are assigned to a Q8 peak, in (c) of FIG. 1, the Q7 peak and Q8 peak have a COSY peak (a matrix element is 1), so that there should be a COSY peak present between peaks assigned to Q7 and peaks assigned to Q8. However, referring to (d) of FIG. 1, there is no COSY peak present between K11 and K13 groups and K8, K9, and K10 groups, such assignment is an unsuitable assignment.

### (2-3) Similarity Calculation Step of Two Spectra (S63)

According to the above-described procedure, peaks Qi (i=1, 2, . . . , M) (referred to as query peaks) of a candidate spectrum are assigned to peaks Kj (j=1, 2, . . . , N) (referred to as key peaks) of the key spectrum, and then similarity between the candidate spectrum and the key spectrum is calculated to derive an optimal similar candidate spectrum with the highest similarity.

The similarity is calculated by the objective function of Equation 2 below, and the optimal similar candidate spectrum with the highest similarity is one that reduces an objective function value in Equation 2.

$$f = {\sum }_{i}^{M} ( {\sum }_{j}^{N} ( {x}_{ij} \cdot {\vert {shift}_{query} ( i ) - {shift}_{key} ( j ) \vert}^{2} ) ) \qquad [ Equation 2 ]$$

(M and N are respectively the number of peaks of the query or candidate spectrum and the key spectrum, X is a binary matrix of M×N, and if the query peak Qi is not assigned to the key peak Kj, Xᵢⱼ=0, and if the query peak Qi is assigned to the key peak Kj, Xᵢⱼ=1. shift_{query}(i) and shift_{key}(j) respectively represent chemical shifts of Qi and Ki.

Meanwhile, the calculation of the objective function has the following restrictions.
\textcircled{1}

$${\sum }_{i}^{M} {X}_{ij} \cdot {intg}_{query} ( i ) = {intg}_{key} ( j )$$

(intg_{query}(i) is an integral value of the query peak Qi, and intg_{key}(j) is an integral value of the key peak Kj)
\textcircled{2} If the multiplicity of Kj is different from the multiplicity of Qi, Xᵢⱼ=0
\textcircled{3} If an integral value of Qi is greater than an integral value of Kj, Xᵢⱼ=0
\textcircled{4} For two key peaks Ki_1 and Ki_2 having a COSY peak, there should be at least one COSY peak present between a set of all query peaks C_query(Ki_1) assigned to Ki_1 and a set of all query peaks C_query(Ki_2) assigned to Ki_2.

As such, while applying the above-described constraints, similarity between two spectra is calculated by calculating the objective function according to Equation 2 above.

### (3) Target Substance Molecular Structure Confirmation Step (S70)

The molecular structure of a candidate substance corresponding to a candidate spectrum derived as most similar in the spectrum similarity calculation procedure is determined as the molecular structure of the target substance.

### (3-1) Final Candidate Molecular Structure Determination Step (S71)

A candidate spectrum with the smallest objective function calculation value to be described below is determined as an optimal similar spectrum with the highest similarity. There is a final candidate molecular structure determination step in which a candidate structure corresponding to the optimal similar spectrum is estimated as the molecular structure of the target substance. The determined final candidate molecular structure is confirmed as the molecular structure of the target substance to be estimated.

### (3-2) Deuterium Substitution Position Estimation Step (S72)

The present disclosure may additionally include a deuterium substitution position estimation step of estimating a deuterium substitution position through spectrum comparison.

If some of hydrogens of a specific molecule are substituted with deuteriums, a 1H NMR peak of the corresponding hydrogen has a unique feature form, and if the deuterium substitution rate is not 100%, the corresponding peak appears in the form of a small peak having an integral value of a decimal point unit rather than an integer, and the corresponding peak appears in a form in which a singlet and a multiplet are mixed. That is, a 1H-NMR peak at the deuterium substitution position has a characteristic shape.

Meanwhile, it is possible to know which hydrogen of a candidate molecular structure corresponds to each peak Qi of a candidate spectrum estimated from a candidate molecular structure, and since each Qi is assigned to each Kj in the above assignment process, it is possible to know a hydrogen assigned to each Kj. Therefore, since it is possible to know a Qi assigned to a deuterium substitution peak having the characteristic shape, it is possible to know which hydrogen of the candidate molecular structure has finally been substituted with a deuterium. When performing a deuterium substitution reaction, the deuterium substitution position may be used to confirm whether the substitution has occurred at a desired position.

By applying the above principle, the deuterium substitution position estimation step finds a peak in which a singlet and a multiplet are mixed among peaks Kj of the key spectrum, finds a peak corresponding to a Qi of the optimal similar spectrum, and finds a corresponding hydrogen in the final estimated candidate molecular structure to identify the position of a hydrogen substituted with a deuterium in the molecular structure of the target substance.

### 2. System for Estimating Molecular Structure of Target Substance According to the Present Disclosure

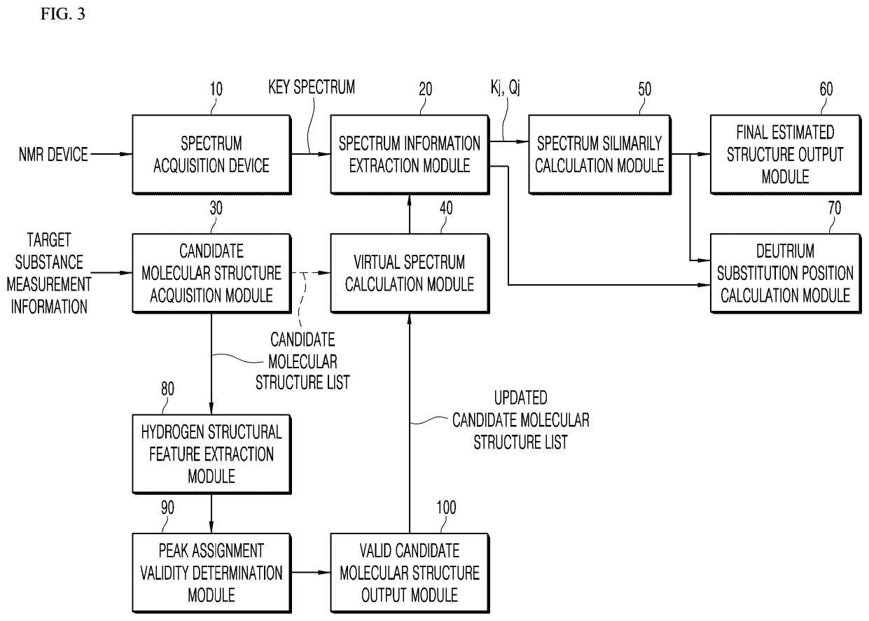

Below, with reference to FIG. 3, a molecular structure estimation system for estimating the molecular structure of a target substance according to the present disclosure will be described.

### (1) Spectrum Acquisition Device (10)

This is a composition that acquires a 1H-NMR spectrum and a 1H-1H NMR spectrum of the target substance from an NMR device. It may be composed of an input unit of a computer input device that receives spectrum information from a typical NMR device. At this time, spectrum of the target substance is called a key spectrum.

### (2) Spectrum Information Extraction Module (20)

It is a composition that acquires spectrum information from the spectrum. The acquired spectrum information is the same as described in the spectrum information acquisition step. A spectrum information extraction module acquires key spectrum information including information of a key spectrum peak Ki from a key spectrum, and acquires candidate spectrum information including information of a candidate spectrum peak Qi of a candidate spectrum.

### (3) Candidate Molecular Structure Acquisition Module (30)

It is a composition that receives results of such as mass spectrometry of the target substance and estimates and calculates a candidate molecular structure of the target substance. Candidate molecular structures, as described above, may be acquired through a method such as mass spectrometry.

In the present disclosure, obtaining a candidate molecular structure which may be the molecular structure of the target substance uses a known method, so that a candidate molecular structure acquisition module of the present disclosure may acquire a molecular weight and a molecular formula of the target substance from an LCMS/MS analyzer and search the molecular weight and the molecular formula in a known database to acquire a candidate molecular structure, or may be composed of a data input module that receives a candidate molecular structure obtained by a known method as described above.

### (4) A Virtual Spectrum Calculation Module (40)

A virtual spectrum calculation module calculates a virtual spectrum for each candidate molecular structure of the target substance calculated by the candidate molecular structure acquisition module. The spectrum of each candidate molecular structure calculated by the virtual spectrum calculation module is input to the spectrum information extraction module 20 to extract candidate spectrum information. The virtual spectrum calculation module 40 is composed of a known virtual spectrum information calculation device or software that calculates virtual spectrum information when molecular structure information is input.

### (5) Spectrum Similarity Calculation Module (50)

A spectrum similarity calculation module matches the peak Qi of the candidate spectrum with the peak Kj of the key spectrum calculated by the spectrum information extraction module 20, and calculates similarity between the key spectrum and the candidate spectrum.

The matching method of Kj and Qi and the spectrum similarity calculation method are the same as described in 2. (2) and 2. (3) above. It is the spectrum with the highest similarity to a candidate spectrum for which the objective function of Equation 1 is calculated to be lowest.

### (6) Final Estimated Structure Output Module (60)

A candidate structure output module determines a candidate molecular structure corresponding to the corresponding candidate spectrum from the optimal similarity spectrum calculated by the spectrum similarity calculation module 50 above as a final estimated structure for the target substance and outputs the corresponding molecular structure information.

### (7) Deuterium Substitution Position Calculation Module (70)

This is a module that determines the candidate molecular structure having the optimal similarity spectrum calculated by the spectrum similarity calculation module as the molecular structure of the target substance, and identifies and calculates the position of a hydrogen substituted by a deuterium in the corresponding molecular structure.

This is to find a peak in which a singlet and multiplet are mixed among the peaks Kj of the key spectrum, finds a peak corresponding to a Qi of the optimal similar spectrum, and finds a corresponding hydrogen in the final estimated molecular structure of the corresponding candidate molecular structure to identify the position of a hydrogen substituted with a deuterium in the molecular structure of the target substance.

### (8) Hydrogen Structural Feature Extraction Module (80)

This is a module that identifies and extracts structural feature information of hydrogen from each candidate molecular structure of the target substance calculated by the candidate molecular structure acquisition module. The structural feature information to be identified is, as described above, the number of isomorphic hydrogens, the number of hydrogens linked to the 1^{st}-neighbor atom, the number of hydrogens linked to the 2^{nd}-neighbor, and identification information for other hydrogens that may have COSY peaks.

As such, by extracting the structural feature information of each hydrogen from each candidate molecular structure, it is determined whether it is possible to assign each hydrogen of the candidate molecular structure to each peak of the spectrum based on the feature information of the hydrogen of each candidate molecular structure in the peak assignment validity determination module.

The hydrogen structural feature extraction module of the present disclosure may be composed of a data input module that identifies the structural feature information of hydrogen using a known algorithm for extracting hydrogen structural information or receives the hydrogen structural feature information identified using the known algorithm.

### (9) Peak Assignment Validity Determination Module (90)

A peak assignment validity determination module is a module that determines, according to Equation 1 above, whether each hydrogen may be assigned to each peak of the spectrum based on the structural feature information of each hydrogen extracted above from the candidate molecular structure. The peak assignment validity determination module is composed of software that obtains an optimal solution according to Equation 1 described above.

The peak assignment validity determination module determines that if the optimal solution according to Equation 1 is not obtained, the corresponding candidate molecular structure is not suitable for the given spectrum.

### (10) Valid Candidate Molecular Structure Output Module (100)

The valid candidate structure output module excludes candidate molecular structures not suitable for the given spectrum as a result of verification in each of the hydrogen structural feature extraction module and the peak assignment validity determination module for the candidate molecular structures calculated above by the candidate molecular structure acquisition module, and outputs a list of the remaining candidate molecular structures as a candidate molecular structure list. That is, the valid candidate molecular structure output module updates the candidate molecular structure list by using the results of the peak assignment validity determination module.

Typically, in order to determine the suitability of a candidate molecular structure, each hydrogen position of each candidate molecular structure is compared with the shape of each peak of a spectrum to determine whether the candidate molecular structure is a valid structure, so that as a molecular structure becomes larger and more complex, the difficulty and required time increase exponentially. However, according to the present disclosure, by converting hydrogen, which may be distinguished by a predetermined rule, into a problem of solving an equation by optimizing only whether or not the hydrogen may correspond to each peak of an NMR spectrum of the target substance, it is possible to very quickly determine the suitability of a candidate molecular structure, and it is also possible to accurately and quickly determine the suitability for a large aromatic compound by utilizing 1H-NMR information as well as 2D 1H-1H NMR information.

In addition, typically, when comparing an actual spectrum with a virtual spectrum for molecular structure estimation of an unknown substance, a method has been used in which the similarity is evaluated higher as signals of similar intensities are positioned at similar X-axis positions. The above typical method is not accurate in determining similarity since the position and intensity of peaks are not significantly different from each other, when a large number of peaks are concentrated in a small region, as in the case of an aromatic macromolecule, and when candidate molecular structures are similar to the extent that only the substitution positions of functional groups are different.

Accordingly, the present disclosure compares the spectra of candidate molecular structures by utilizing not only x and y coordinates of a 1H-NMR spectrum peak, but also multiplicity and 2D 1H-1H COSY NMR information, so that it has become possible to build a system that accurately determines the similarity of candidate structures for aromatic polymers.

1. A method for predicting a molecular structure of a target substance from a 1H-NMR spectrum and a 1H-1H COSY spectrum of the target substance, the method comprising:
acquiring a ¹H-NMR spectrum and a 1H-1H COSY spectrum of a target substance as a key spectrum;
acquiring a peak position, a peak split value, a peak integral value, and COSY information of the key spectrum to obtain key spectrum information;
acquiring molecular structure information of a plurality of candidates of the target substance;
creating a candidate molecular structure list including the acquired molecular structure information of the plurality of candidates;
acquiring a virtual spectrum corresponding to each candidate molecular structure from the candidate molecular structure list as a spectrum of each candidate molecular structure and acquiring candidate spectrum information thereof;
comparing the key spectrum information with each candidate spectrum information to calculate a similarity between the key spectrum and each candidate spectrum; and
determining the target substance by determining the candidate spectrum having the highest similarity with the key spectrum, and selecting the molecular structure of the corresponding candidate substance as the molecular structure of the target substance.

2. The method of claim 1, further comprising,
after creating the candidate molecular structure list,
extracting structural feature information of each hydrogen from each of the plurality of candidates in the candidate molecular structure list;
determining peak assignment possibility by verifying whether all hydrogens of each of the plurality of candidates in the candidate molecular structure list may be assigned to all peak positions of the key spectrum of the target substance, based on the structural feature information of each hydrogen; and
updating of the candidate molecular structure list by leaving only the candidate molecular structures which may assign all of the above hydrogens to all peak positions of the key spectrum and excluding the remaining candidate molecular structures from the candidate molecular structure list.

3. The method of claim 2, wherein determining peak assignment possibility
obtains an optimal solution to Equation 1, and when the optimal solution is obtained, determines the same as a candidate molecular structure, and updating step updating the candidate molecular structure list excludes unsuitable candidate molecular structures, wherein Equation 1 is defined by:
$$minimize {\sum }_{i}^{N} ( {s}_{i} + {t}_{i} ) \qquad ( Equation 1 )$$
for M×N sized matrix X
(If hydrogen j is not assigned to peak i, Xᵢⱼ=0, if hydrogen j is assigned to peak i, Xᵢⱼ=1, j=1, 2, . . . , M, i=1, 2, . . . , N), wherein Equation 1 is constrained by:
\textcircled{1} sᵢ≥0, tᵢ≥0 for 1≤i≤N,
\textcircled{2} For all 1≤i≤N, the peak integral value of hydrogen j is intg_{hydrogen}(j), and the integral value of peak i of the key spectrum is intgₚₑₐₖ(i),
$${s}_{i} = {intg}_{peak} ( i ) - {\sum }_{j}^{M} {X}_{ij} \cdot {intg}_{hydrogen} ( j ) .$$
$${t}_{i} = {intg}_{peak} ( i ) + {\sum }_{j}^{M} {X}_{ij} \cdot {intg}_{hydrogen} ( j )$$
\textcircled{3} Xᵢⱼ=0, (if the multiplicity of hydrogen j is different from the multiplicity of peak i)
\textcircled{4} Xᵢⱼ=0, (if the peak integral value of hydrogen j is greater than the integral value of peak i)
\textcircled{5} For two peaks i_1 and i_2 having COSY peaks, there should be at least one COSY peak present between all hydrogen sets C_hydrogen(i1) assigned to the i_1 and all hydrogen sets C_hydrogen(i2) assigned to the i_2.

4. The method of claim 1, wherein the key spectrum information and the candidate spectrum information comprise:
a COSY peak, which is a pair of peak A and peak B, wherein the peak A is each peak of each spectrum and the peak B is another peak that is related to the peak A;
a peak integral value, which is the area of each peak;
a position value (hydrogen atom shift value) of each peak; and
a peak split value, which is the number of small peaks constituting each peak.

5. The method of claim 4, wherein
when a peak of the key spectrum is Kj and a peak of the candidate spectrum is Qi, the peaks to satisfy Equation 2 below:
$$Integral value of Qi = < Integral value of Kj \qquad ( Equation 2 )$$
$$Split value of Qi = Split value of Kj .$$

6. The method of claim 5, wherein one or more hydrogens having a COSY relationship with Qi are assigned to another peak having a COSY relationship with Ki.

7. The method of claim 6, wherein determining the target substance further includes:
determining a molecular structure corresponding to a candidate spectrum derived as most similar to the key spectrum among candidate spectra as the molecular structure of the target substance; and
identifying hydrogen of an estimated molecular structure of the target substance corresponding to a peak in which a singlet and a multiplet are mixed among the peaks of the key spectrum as a hydrogen substituted with a deuterium.

8. A method for determining similarity by comparing two NMR spectra, the method comprising:
extracting peak value information for spectrum comparison from each peak of a first spectrum as a key spectrum and a second spectrum as a candidate spectrum;
matching each peak of the first spectrum and the second spectrum;
calculating similarity between the first spectrum and the second spectrum by comparing the peak value information of the peaks of the first spectrum and the second spectrum matched.

9. The method of claim 8, wherein the peak value information comprises:
a COSY peak, which is a pair of peak A and peak B, wherein the peak A is each peak of each spectrum and the peak B is another peak that is related to the peak A;
a peak integral value, which is the area of each peak;
a position value (hydrogen atom shift value) of each peak; and
a peak split value, which is the number of small peaks constituting each peak.

10. The method of claim 9, wherein the peak correspondence step matches each peak to satisfy Equation 3 below:
$$Integral value of Qi = < Integral value of Kj \qquad ( Equation 3 )$$
$$Split value of Qi = Split value of Kj .$$

11. The method of claim 9, wherein
calculating similarity between the first spectrum and the second spectrum is calculated by an objective function of Equation 4 below:
$$f = {\sum }_{i}^{M} ( {\sum }_{j}^{N} ( {X}_{ij} \cdot {\vert {shift}_{query} ( i ) - {shift}_{key} ( j ) \vert}^{2} ) ) \qquad ( Equation 4 )$$
(M and N are respectively numbers of peaks of the candidate spectrum and the key spectrum, X is a binary matrix of M×N, and when a query peak Qi is not assigned to a key peak Kj, Xᵢⱼ=0, and when the query peak Qi is assigned to the key peak Kj, Xᵢⱼ=1, wherein shift_{query}(i) and shift_{key}(j) respectively represent chemical shifts of Qi and Ki, and Equation 4 is constrained by:
\textcircled{1}
$${\sum }_{i}^{M} {X}_{ij} \cdot {intg}_{query} ( i ) = {intg}_{key} ( j ) \text{?} ral$$
$$\text{?} \text{indicates text missing or illegible when filed}$$
value of the query peak Qi, and intg_{key}(j) is an integral valu [symbol]
\textcircled{2} When the multiplicity of Kj is different from the multiplicity of Qi, Xᵢⱼ=0
\textcircled{3} When an integral value of Qi is greater than an integral value of Kj, Xᵢⱼ=0
\textcircled{4} When two key peaks Ki_1 and Ki_2 having a COSY peak, there is at least one COSY peak present between a set of all query peaks C_query(Ki_1) assigned to Ki_1 and a set of all query peaks C_query(Ki_2) assigned to Ki_2.

12. A system for estimating a molecular structure of a target substance, the system comprising:
a spectrum acquisition unit that acquires a ¹H-NMR spectrum and a 1H-¹H NMR spectrum of a target substance from an NMR device as a key spectrum of the target substance;
a candidate molecular structure acquisition module that acquires candidate molecular structures of the target substance and calculates a candidate molecular structure list;
a virtual spectrum calculation module that calculates a virtual spectrum as a candidate spectrum from each candidate molecular structures included in the candidate molecular structure list;
a spectrum information extraction module that extracts spectrum information from each of the key spectrum and each candidate spectrum;
a spectrum similarity calculation module that calculates similarity between the key spectrum and each candidate spectrum by using the spectrum information of the key spectrum and each candidate spectrum; and
a final estimated structure output module that outputs the candidate molecular structure corresponding to the candidate spectrum with the highest similarity as a final estimated molecular structure of the target substance.

13. The system of claim 12, further comprising:
a hydrogen structural feature extraction module that identifies and extracts structural feature information of each hydrogen from each candidate molecular structure of the target substance;
a peak assignment validity determination module that verifies, based on the structural feature information of each hydrogen identified from the candidate molecular structure, whether each hydrogen of each candidate molecular structure may be assigned to all peaks in the key spectrum; and
a valid candidate molecular structure output module that updates the candidate molecular structure list by using results of the peak assignment validity judgment module.

14. The system of claim 12, wherein the spectrum information extracted from the spectrum information extraction module comprises the following information:
(a) Peak position (chemical shift value): x-axis value of each peak in one-dimensional spectrum
(b) Peak split value (multiplicity (split)): Number of small peaks forming each peak (for example, in the spectrum of FIG. 1(a), the split of the leftmost peak is 2, and the split of the rightmost peak is 3)
(c) Peak integral value: Area value of each peak
(d) COSY information (COSY): Information of all peak pairs in which COSY peaks are observed.

15. The system of claim 14, wherein the similarity calculation module calculates the similarity between the key spectrum and the candidate spectrum by using an objective function in Equation 5 below:
$$f = {\sum }_{i}^{M} ( {\sum }_{j}^{N} ( {X}_{ij} \cdot {\vert {shift}_{query} ( i ) - {shift}_{key} ( j ) \vert}^{2} ) ) \qquad ( Equation 5 )$$
(M and N are respectively numbers of peaks of the candidate spectrum and the key spectrum, X is a binary matrix of M×N, and when a query peak Qi is not assigned to a key peak Kj, Xᵢⱼ=0, and when the query peak Qi is assigned to the key peak Kj, Xᵢⱼ=1, wherein shift_{query}(i) and shift_{key}(j) respectively represent chemical shifts of Qi and Ki, and Equation 5 is constrained by:
\textcircled{1}
$${\sum }_{i}^{M} {X}_{ij} \cdot {intg}_{query} ( i ) = {intg}_{key} ( j ) \text{?} gral$$
$$\text{?} \text{indicates text missing or illegible when filed}$$
value of the query peak Qi, and intg_{key}(j) is an integral val [symbol]
\textcircled{2} When the multiplicity of Kj is different from the multiplicity of Qi, Xᵢⱼ=0
\textcircled{3} When an integral value of Qi is greater than an integral value of Kj, Xᵢⱼ=0
\textcircled{4} When two key peaks Ki_1 and Ki_2 having a COSY peak, there is at least one COSY peak present between a set of all query peaks C_query(Ki_1) assigned to Ki_1 and a set of all query peaks C_query(Ki_2) assigned to Ki_2.

16. The system of claim 15, further comprising a deuterium substitution position calculation module that finds a Qi of a candidate spectrum of a final candidate molecular structure assigned to Kj in which a singlet and a multiplet are mixed among the peaks of the key spectrum, and identifies a hydrogen of the corresponding final candidate molecular structure as a hydrogen substituted with a deuterium.

17. The method of claim 2, wherein the key spectrum information and the candidate spectrum information comprise:
a COSY peak, which is a pair of peak A and peak B, wherein the peak A is each peak of each spectrum and the peak B is another peak that is related to the peak A;
a peak integral value, which is the area of each peak;
a position value (hydrogen atom shift value) of each peak; and
a peak split value, which is the number of small peaks constituting each peak.

18. The method of claim 3, wherein the key spectrum information and the candidate spectrum information comprise:
a COSY peak, which is a pair of peak A and peak B, wherein the peak A is each peak of each spectrum and the peak B is another peak that is related to the peak A;
a peak integral value, which is the area of each peak;
a position value (hydrogen atom shift value) of each peak; and
a peak split value, which is the number of small peaks constituting each peak.

19. The system of claim 13, wherein the spectrum information extracted from the spectrum information extraction module comprises the following information:
(a) Peak position (chemical shift value): x-axis value of each peak in one-dimensional spectrum
(b) Peak split value (multiplicity (split)): Number of small peaks forming each peak (for example, in the spectrum of FIG. 1(a), the split of the leftmost peak is 2, and the split of the rightmost peak is 3)
(c) Peak integral value: Area value of each peak
(d) COSY information (COSY): Information of all peak pairs in which COSY peaks are observed.

20. The system of claim 19, wherein the similarity calculation module calculates the similarity between the key spectrum and the candidate spectrum by using an objective function in Equation 5 below:
$$f = {\sum }_{i}^{M} ( {\sum }_{j}^{N} ( {X}_{ij} \cdot {\vert {shift}_{query} ( i ) - {shift}_{key} ( j ) \vert}^{2} ) ) \qquad ( Equation 5 )$$
(M and N are respectively numbers of peaks of the candidate spectrum and the key spectrum, X is a binary matrix of M×N, and if when a query peak Qi is not assigned to a key peak Kj, Xᵢⱼ=0, and when the query peak Qi is assigned to the key peak Kj, Xᵢⱼ=1, wherein shift_{query}(i) and shift_{key}(j) respectively represent chemical shifts of Qi and Ki, and Equation 5 is constrained by:
\textcircled{1}
$${\sum }_{i}^{M} {X}_{ij} \cdot {intg}_{query} ( i ) = {intg}_{key} ( j ) \text{?} gral$$
$$\text{?} \text{indicates text missing or illegible when filed}$$
value of the query peak Qi, and intg_{key}(j) is an integral val [symbol]
\textcircled{2} When the multiplicity of Kj is different from the multiplicity of Qi, Xᵢⱼ=0
\textcircled{3} When an integral value of Qi is greater than an integral value of Kj, Xᵢⱼ=0
\textcircled{4} When two key peaks Ki_1 and Ki_2 having a COSY peak, there is at least one COSY peak present between a set of all query peaks C_query(Ki_1) assigned to Ki_1 and a set of all query peaks C_query(Ki_2) assigned to Ki_2.
